SEQUENCE LISTING (2 sequences)

SEQ ID NO: 1 - DNA, 23 bp, Artificial Sequence
  cyclin B1 siRNA sense
acaugagagccauccuaauugtt

SEQ ID NO: 2 - DNA, 23 bp, Artificial Sequence
  cyclin B1 siRNA anti-sense
caauuaggauggcucucaugutt

(19) United States
(12) Patent Application Publication
(10) Pub. No.: US 2012/0052573 A1
(43) Pub. Date: Mar. 1, 2012

(54) ONE-POT SYNTHESIS OF LINEAR-LIKE POLYETHYLENIMINE FOR INTRACELLULAR IMAGING AND NUCLEIC ACID DELIVERY

(75) Inventors: Shu-Yi LIN, Hsinchu (TW); Fong-Sian LIN, Taichung County (TW); Meng-Kai CHEN, Hsinchu (TW); Yu-Chen JAO, Miaoli County (TW); Lin-Ren TSAI, Taipei County (TW); Hong-Yi LIN, Taoyuan County (TW); Chung-Shi YANG, Taichung (TW); Yeu-Kuang HWU, Taipei (TW)

(73) Assignee: NATIONAL HEALTH RESEARCH INSTITUTES, Miaoli County (TW)

(21) Appl. No.: 12/868,939

(22) Filed: Aug. 26, 2010

(51) Int. Cl. C12N 5/09; A61K 31/7088; C08G 73/00

(52) U.S. Cl. 435/375; 514/44 R; 204/157.81; 528/424

(57) ABSTRACT

A method of synthesizing polyethyleneimine with a substantially linear backbone is disclosed. The method comprises exposing ethylenediamine dissolved in a solution to electromagnetic radiation for a sufficient time to polymerize the ethylenediamine and thereby resulting in formation of polyethylenimine with a substantially linear backbone in the solution. A method of synthesizing a homopolymer with a substantially linear backbone is also disclosed. In addition, a composition comprising polyethylenimine synthesized from the aforementioned method is disclosed, in which the polyethylenimine comprises a backbone conformation that is substantially linear and has a distribution of molecular weights (MW) ranging from 1 kDa to 200 kDa; and the polyethyleneimine has no cytotoxicity at a concentration of 12 μg/ml.

### FIELD OF THE INVENTION

The present invention relates generally to synthesis of polyethyleneimine, and more specifically to synthesis of linear-like polyethyleneimine in one reactor.

### BACKGROUND OF THE INVENTION

Polyethyleneimine (PEI) is one of the promising nonviral vectors. PEI can form a cationic complex by associating with nucleic acids such as siRNA, and the complex can follow a well-known endosome-escape mechanism for efficient mRNA silencing. Most of the reported PEIs are branch-like (BPEIs), and their delivery efficiency is strongly related to their molecular weights. BPEIs with high molecular weights can enhance efficient delivery, but they also induce significant cell death as compared to those with low molecular masses. Alternatively, linear-like PEIs (LPEIs) with high molecular weights were demonstrated to have minimal toxicities and inflammatory responses. Several strategies have been established based on the grafting modification of LPEI to enhance delivery power for mRNA silencing. LPEI has thus attracted attention as an emerging vehicle for siRNA delivery because it has much less adverse side effects than BPEI. In addition, LPEI exhibits blue photoluminescence, and may be used as a powerful labeling-free probe as cellular trackers. The traditional protocol for LPEI synthesis is a stepwise polymerization. The cationic oxazoline compounds are prepared as monomers, which undergo a ring-opening reaction to activate chain propagation and then termination by acid hydrolysis. The oxazoline compounds were prepared in organic solvents such as dimethylformamide, which is hazardous and toxic for cells. Thus, residual chemicals such as solvents and acids from a prerequisite purification may cause unexpected results in the subsequent biological experiments.

Therefore, a heretofore unaddressed need exists in the art to address the deficiencies and inadequacies, especially in connection with development of a high-throughput and organic solvent free protocol for LPEI synthesis for a wide range of applications.

### SUMMARY OF THE INVENTION

In one aspect, the invention relates to a method of synthesizing polyethyleneimine with a substantially linear backbone comprising exposing ethylenediamine dissolved in a solution to electromagnetic radiation for a sufficient time to polymerize the ethylenediamine and thereby resulting in formation of polyethylenimine with a substantially linear backbone in the solution.

In another aspect, the invention relates to a method of synthesizing a homopolymer with a substantially linear backbone, comprising exposing a monomer dissolved in a solution to electromagnetic, radiation for a sufficient time to polymerize the monomer and thereby resulting formation of a homopolymer with a substantially linear backbone in the solution.

Further in another aspect, the invention relates to a composition comprising polyethyleneimine with a substantially linear backbone synthesized from exposing ethylenediamine to electromagnetic radiation for a sufficient time according to the aforementioned method; wherein the polyethyleneimine comprises a backbone conformation that is substantially linear and has a distribution of molecular weights (MW) ranging from 1 kDa to 200 kDa; and wherein the polyethyleneimine has no cytotoxicity at a concentration of 12 μg/ml.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and together with the written description, serve to explain the principles of the invention. Wherever possible, the same reference numbers are used throughout the drawings to refer to the same or like elements of an embodiment.

### BRIEF DESCRIPTION OF THE DRAWINGS

FIG. 1 is a schematic drawing showing a plausible polymerization mechanism for LPEI (b).

FIGS. 2A shows ¹H NMR spectra of starting material (upper panel; *: compound a) and compound b (lower panel).

FIG. 2B shows ¹³C NMR spectra of starting material (upper panel; *: compound a) and compound b (lower panel).

FIG. 2C is a photograph showing the results of PAGE gel electrophoresis of compound b. Lane M1: protein marker illuminated with white light; Lane M2; the same protein marker illuminated with UV-light; Lane L: compound b illuminated with white light.

FIG. 2D is a conformation plot for compound b (S₁), PEG (S₂) and dendrimer (S₃), respectively,

FIGS. 3A-3B are graphs showing confocal microscope images of intracellular delivery. Scale bar: 30 μm.

FIG. 3C is a graph showing cytotoxicity evaluation of compound b.

FIGS. 4A-4E are graphs showing cell cycle arresting analysis.

FIG. 5A is a graph showing UV-Vis (dashed line) and emission spectra (solid line) of compound b.

FIG. 5B is a graph showing photo luminescent lifetime of compound b.

FIG. 6 is a graph showing a MALDI-TOF spectrum of compound b.

FIG. 7A is a graph showing emission spectra of LPEIs (compound b) formed in a solution gassed with N₂ (solid line) or O₂ (dash line) during the synthesis.

FIG. 7B shows ¹H spectra (400 MHz) of compounds b formed without oxygenation (upper panel) and with oxygenation (lower panel).

FIG. 8 shows ¹H spectra of commercial branch-like polyethylenimine (BPEI).

FIGS. 9A-9B are graphs showing titration curves and differential curves of compound b, respectively.

FIG. 10 is a graph showing fluorescent emission spectra of compound b in water, in which the synthesis of the compound b was carried out with air or N2(g)-bubbling fin a time period as indicated.

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. As used in the description herein and throughout the claims that follow, the meaning of “a”. “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention. Additionally, some terms used in this specification are more specifically defined below.

### DEFINITIONS

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain may be highlighted, for example using italics and/or quotation marks. The use of highlighting has no influence on the scope and meaning of a term; the scope and meaning of a term is the same, in the same context, whether or not is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, when a number or a range is recited, ordinary skill in the art understand it intends to encompass an appropriate, reasonable range for the particular field related to the invention.

As used herein, “dendrimers” are regular, highly branched monomers leading to a monodisperse, tree-like or generational structure.

As used herein, a “linear polymer” is a polymer whose molecule is arranged in a chainlike fashion with few branches or bridges between the chains. Linear polyethyleneimine (PEI) contains all secondary amines, in contrast to branched PEIs which contain primary, secondary and tertiary amino groups. The linear PEI is solid at room temperature where branched PEI is liquid at all molecular weights.

The term “homopolymer” shall generally refer to a polymer formed from a single monomer.

The terms Poly(ethyleneimine) and Poly(ethylenimine) are interchangeable.

The invention relates to linear-like and photoluminescent polyethyleneimine (LPEI) that may be synthesized in a one-pot reaction within about 5 min upon synchrotron radiation. The LPEI was of a minimal cytotoxicity concern, and can act as a vehicle for efficient siRNA delivery and as a potential intracellular tracker.

In one aspect, the invention relates to a method of synthesizing polyethyleneimine with a substantially linear backbone comprising exposing ethylenediamine dissolved in a solution to electromagnetic radiation for a sufficient time to polymerize the ethylenediamine and thereby resulting in formation of polyethylenimine with a substantially linear backbone in the solution.

Without intent to limit the scope of the invention, an example of substantially branched (or branch-like) polymer is a polymer with a slope value lower than 0.5-0.6, and a substantially linear (or linear-like) polymer shall generally refers to a polymer with a slope value higher than 0.5-0.6 on the base of the conformation plot of log r_{g} vs. log Mw.

The method may further comprise removing ethylenediamine dissolved in the solution after formation of the polyethyleneimine with a substantially linear backbone.

The reactive solution (i.e., ethylenediamine solution) is not captured in any liquid chamber. The radiation is passed to the reactive solution without passing through a means for screening.

The reactive solution is without organic solvents, acids, free-radical-forming agents, azo initiators, or peroxide initiators, or all of the aforementioned agents.

In one embodiment of invention, the method synthesizes polyethyleneimine that is substantially free of branched chains.

In one embodiment of invention, the ethylenediamine is irradiated at a temperature between 4° C. and 60° C.

In another embodiment of the invention, the ethylenediamine is irradiated at a temperature between 4° C. and 50° C., between 4° C. and 40° C., between 4° C. and 30° C., or between 4° C. and 25° C.

In another embodiment of the invention, the ethylenediamine is irradiated for no more than 10 minutes.

In another embodiment of the invention, the polyethylenimine with a substantially linear backbone is formed without reagent selected from organic solvents, acids, and poly(alkyl ethylene).

In another embodiment of the invention, the ethylenediamine solution is not gassed with oxygen during exposure to the electromagnetic radiation.

In another embodiment of the invention, the polyethylenimine with a substantially linear backbone has a distribution of molecular weights ranging from 1 kDa to 200 kDa, 1 kDa to 190 kDa, 1 kDa to 180 kDa, 1 kDa to 170 kDa, 1 kDa to 160 kDa, 1 kDa to 150 kDa, 1 kDa to 140 kDa, 1 kDa to 130 kDa, 1 kDa to 120 kDa, 1 kDa to 110 kDa, from 1 kDa to 100 kDa, from 1 kDa to 90 kDa, from 1 kDa to 80 kDa, from 1 kDa to 70 kDa, from 1 kDa to 60 kDa, from 1 kDa to 50 kDa, from 1 kDa to 40 kDa, from 1 kDa to 30 kDa, or from 1 kDa to 22 kDa.

In another embodiment of the invention, the polyethyleneimine with a substantially linear backbone has a distribution of molecular weights ranging from 3 kDa to 15 kDa.

In another embodiment of the invention, the electromagnetic radiation is selected from X-rays, microwaves, and gamma-rays.

In another embodiment of the invention, the electromagnetic radiation comprises X-rays.

In another embodiment of the invention, the radiation has energy of 4 KeV to 3,000 KeV and a radiation dose of from 2×10³ to 10⁷ Gy/s.

In another embodiment of the invention, the electromagnetic radiation has energy of 4 to 100 KeV and a radiation dose of 10⁴ to 10⁶ Gy/s.

In another embodiment of the invention, the polyethylenimine with a substantially linear backbone is formed with stirring.

Further in another embodiment of the invention, the polyethylenimine with a substantially fin backbone is formed without stirring.

In another aspect, the invention relates to a method of synthesizing a homopolymer with a substantially linear backbone, comprising exposing a monomer dissolved in a solution to electromagnetic radiation for a sufficient time to polymerize the monomer and thereby resulting in formation of a homopolymer with a substantially linear backbone in the solution.

In another embodiment of the invention, the monomer and homopolymer are without double and triple bonds; and wherein the method is without the step of precipitating the homopolymer from a polymer radical; and further wherein the radiation is unfiltered, through a means for screening or is unfiltered through a radiation mask.

Further in another aspect, the invention relates to a composition comprising polyethyleneimine with a substantially linear backbone synthesized from exposing ethylenediamine to electromagnetic radiation for a sufficient time according to the aforementioned method; wherein the polyethyleneimine comprises a backbone conformation that is substantially linear and has a distribution of molecular weights (MW) ranging from 1 kDa to 200 kDa; and wherein the polyethylenimine has no cytotoxicity at a concentration of 12 μg/ml.

In one embodiment of the invention, the composition comprises polyethyleneimine having a concentration ranging from 100 ng/ml to 10⁶ ng/ml; and wherein the polyethyleneimine has no significant cytotoxicity in the concentration range indicated.

In another embodiment of the invention, the composition comprises polyethyleneimine having a concentration ranging from 100 ng/ml to 10⁵ ng/ml; and wherein the polyethyleneimine has no significant cytotoxicity in the concentration range indicated.

Further in another embodiment of the invention, the composition further comprises a nucleic acid.

Yet in another embodiment of the invention, the nucleic acid is a small interfering RNA (siRNA).

### EXAMPLES

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Methods and Materials

Synthesis of compound b. All chemicals including ethylenediamine (compound a) were purchased from SIGMA-ALDRICH® (St. Louis, USA). To synthesize compound b, the monomer (100 μL) was added to 5 mL of water (18 MΩ¹) and then irradiated with X-rays at room temperature for 5 min to 10 min. The final products were dried by lyophilizer or distilled by Kugerlrohr (BÜCHI) to remove extra starting material. Compound b was 17 mg (18.9%). Both ¹H NMR and ¹³C NMR were recorded in 80% D₂O (4.80 ppm) with the nuclear magnetic resonance spectrometer Varian 400-MR system. See Shu-Yi Lin, et al., “One-pot synthesis of linear-like and photoluminescent polyethyleneimine for intracellular imaging and siRNA delivery” Chem. Commun. 2010. 46, 5554-5556; and Supplemental materials thereof, both of which are herein incorporated by reference in their entireties.

Cellular uptake. A human lung cancer cell line, H460, was cultured in a humidified atmosphere with 5% CO₂. The cell culture medium was RPMI 1640 (GIBCO®), supplemented with 10% fetal bovine serum (FIBS; GIBCO®). For confocal microscopy, cells were plated 24 h before each experiment. After incubation with LPEI (compound b) for 1.5 h, cells were stained with the nucleus-specific dye SYTO® 59. Images were captured with an Olympus FV10i confocal spectral microscope using 60× oil immersion objective.

Flow cytometry for cell cycle analysis. Human lung cancer cells, H460, at 2×10⁵ cells/mL were treated with cyclin B1siRNA alone (200 nM) or compound b/siRNA complex ([compound b]=100 ng/mL, [cyclin B1 siRNA]=200 nM) in RPMI 1640 medium for 1 h at 37° C., 5% CO_{2(g)}. After the treatments, cells were washed twice in PBS buffer solution and fixed in cold. PBS solution containing 75% ethanol. After washing with PBS and centrifugation at 1,500 rpm for 5 min, cells were stained with propidium iodide (PI) and analyzed by FACS Calibur (BD PharMingen, N.J., USA) using WinMDI 2.9 analysis software. The cyclic B1 siRNA sequence is 5′-ACAUGAGAGCCAUCCUAAUUGTT-3 (SEQ ID NO: 1) for sense and 5′-CAAUUAGGAUGGCUCUCAUGUTT-3 (SEQ ID NO: 2) (NCBI accession number of cyclin B1: NM031966) for anti-sense.

MTT cell viability assay for cytotoxicity. The proliferation of human lung cancer cells H460 was examined, in the presence of various concentrations of LPEI (compound b) of the invention, commercial PEIs, e.g., BPEI (Mₙ=1800) (Aldrich, Cat. No. 408700), and in the mixture of LPEI and BPEI (Mₙ=423), respectively, using 3-(4,5-dimethylthiazol-2-yl)-2,5-diphenyl tetrazolium bromide (MIT assay, Sigma, Mo., USA). The mixture of LPEI and BPEI (Mₙ=423) was purchased from Sigma-Aldrich (Cat. No. 468533; ethylenimine oligo mixture, average Mn=˜423), which is a mixture of linear and branched chains. Briefly, cells were plated onto 24-well cell culture plates at a density of 2×10⁵ cells/mL per well with a final volume of 200 μL and cultured for overnight. The cells were then treated with LPEI (compound b) or commercial PEI for 48 h at 37° C., 5% CO_{2(g)}. After the treatments, die cells were incubated with MTT at 37° C. for 1 h. After cell lysis, the intracellular formazan product was dissolved in DMSO and quantified by a conventional ELISA reader at 540 nm.

Measurement of quantum yield and lifetime of compound b. Compound b was dissolved in deionized water (DI) water and the fluorescence quantum yield (QY) was compared to quinine (QY=0.53) while the absorbed intensity of the solution containing either compound b or quinine was adjusted to >0.01 and <0.06.

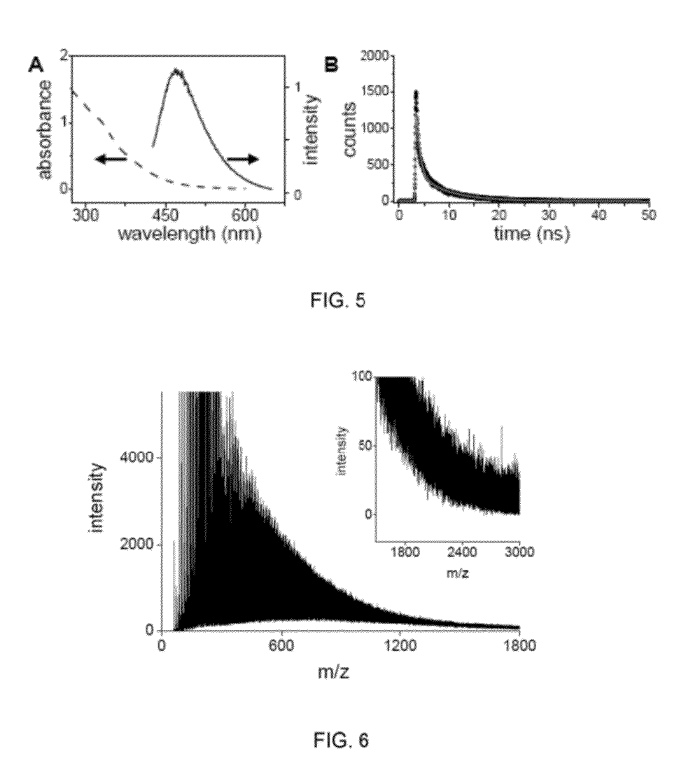

Measurement of the number of secondary amines in compound b. Compound b (0.011 g) was dissolved in a co-solvent (20 mL, isopropyl alcohol:ethylene glycol=1:1). After thorough mixing, the solution was either added acetic anhydride (0.2 mL) to block all primary (1°-amine) and secondary amines (2′-amine) or added salicylaldehyde (0.2 mL) to block only the primary amines, respectively. After reacting for 30 minutes, the solutions were titrated with 0.01N HCl, FIG. 5 shows two titration curves of compound b. Their inflection points of apparent pH versus the volume of hydrochloric acid were re-plotted in FIG. 5B. The percentage of 2°-amine of the polymer compound b was calculated as being approximately 90%.

FIG. 5A shows the optical properties such as UV-Vis (dashed line) and emission spectra (solid line) of compound b, which indicates a maximum emission at 478 nm.

FIG. 5B shows the photoluminescent lifetime of compound b, in which the curve (gray line) has been fitted to a biexpontential decay (black-dot line) after measurement by pulsed diode light source with 405 nm.

### Results

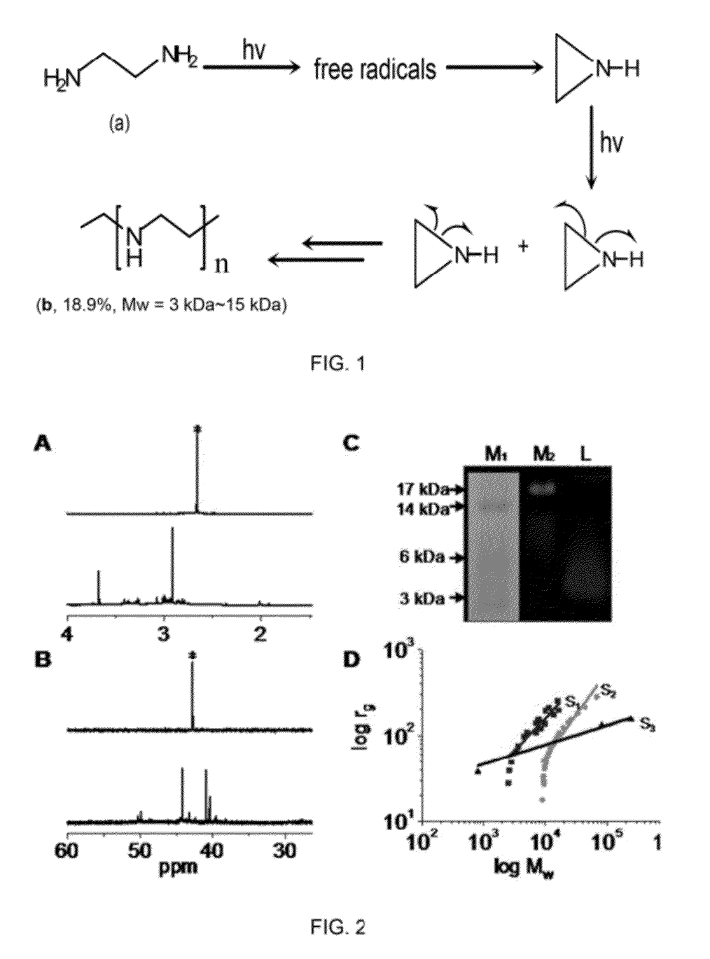

The invention relates to a simple strategy for one-pot (one-step) synthesis of linear-like and photoluminescent polyethyleneimine (PEI) by synchrotron X-ray (4-30 keV, 10⁵ Gy s⁻¹), which is a strong radiation source capable of generating free radicals in the absence of catalysts and chemical initiators. Ethylene diamine (a, 100 μL) was used as a starting material (FIG. 1) and added to a 5 mL aqueous solution. The mixture was irradiated for approximately 5-10 min by synchrotron X-rays. The color of the aqueous solution gradually changed from colorless to pale-yellow. The optical properties of compound b were examined. FIG. 5A displays the absorption and emission spectra of compound b, which shows an apparent absorption band from 250 to 450 nm (dashed line) and an emission maximum appearing, around 478 am. The quantum yield and lifetime of compound b were 2% and 2.6 ns, respectively (FIG. 5B).

FIG. 1 illustrates a plausible polymerization mechanism for LPEI (b),

FIG. 5A shows optical properties including UV-Vis (dashed line) and emission spectra (solid line) of compound b, which indicate a maximum emission appearing at 478 nm. A white light illumination of compound b showed blue color fluorescence. FIG. 5B shows the photoluminescent lifetime of compound b, in which the curve (gray line) has been fitted to a biexpontential decay (black-dot line) after measuring with a pulsed diode light source at 405 nm.

The structure of compound b was examined by ¹H NMR and ¹³C NMR combined with Distortionless Enhancement by Polarization Transfer (DEPT) for determining the presence of primary, secondary and tertiary carbon atoms. The ¹H NMR Spectrum showed two major peaks at 2.83 ppm and 3.65 ppm (compound b; FIG. 2A, bottom panel), which could be assigned to the —CH₂CH₂—NH and —CH₂CH₂NH₂⁺ signals of the ethylenimine units, respectively. The spectrum showed one minor peak at 2.10 ppm (FIG. 2A, bottom panel), which was assigned to the terminal methyl group. Further identification of compound b by ¹³C NMR (FIG. 2B) showed that one peak at 42 ppm from the starting material (*: compound a; FIG. 2B, top panel) had disappeared and several new peaks appeared at 39.5, 40.4, 40.5, 40.9, 43.3, 44.2, 49.9, and 50.3 ppm (compound b: FIG. 2B, bottom panel), which could be assigned to the methylene groups neighboring the amine, indicative of the formation of a PEI backbone structure. The weight-averaged molecular mass (M_{w}) and conformation were estimated by size-exclusion chromatography coupled with a multi-angle light scattering (SEC-MALS). The M_{w} of compound b was approximately 3 kDa and the polydispersity index (PDF) was either 1.29±0.12 or 7.1±5.6, which was dependent on whether the reactant solution had been stirred or not during the X-ray irradiation (1.29 for stirred and 7.1 for unstirred solutions). In addition, the M_{w} had a polydistribution ranging from 3 kDa to 15 kDa, depending on the reactant stoichiometry. For example, increasing the amount of the monomer to 500 μL resulted in polymers with a high M_{w}.

For further confirmation of the M_{w} of compound b, MALDI-TOF mass spectrometry (FIG. 6) and polyacrylamide gel electrophoresis (PAGE; FIG. 2C) were performed. FIG. 6 is a MALDI-TOF spectrum of compound b. It shows indistinct and multiple peaks under 3 kDa, suggesting that the fine structures were from the fragmentation of compound b. On the contrary. FIG. 2C reveals one distinct band between 3 kDa and 6 kDa on PAGE, indicative of the M_{w} of compound b with a narrow distribution. Moreover, the conformation of compound b was studied by SEC-MALS, which could also provide information on the radium of gyration (r_{g}) in addition to molecular mass. FIG. 2D displays a conformation plot of log r_{g} vs. log M_{w} for compound b and two other well-defined polymers, which were a linear-like polyethylene glycol (PEG) and a sphere-like dendrimer. Their slopes from S₁ to S₃ were 0.86±0.04, 0.99±0.02 and 0.3±0.09, respectively. According to a previous report, when slope values were lower or higher than 0.5˜0.6, the conformations were branch-like and linear-like, respectively. To verify the correlation between the conformation and the slope value, a sphere-like dendrimer and a linear-like PEG were used as model compounds. Their slope values were 0.3±0.09 (<0.5˜0.6) and 0.99±0.02 (>0.5˜0.6), respectively, indicating that their conformations were branch-like and linear-like and could be correlated to a previous report. In this study, the slope value (0.86±0.04) of compound b was higher than 0.5˜0.6 indicative of forming a linear-like conformation.

FIGS. 2A and 2B show ¹H NMR and ¹³C NMR spectra, respectively, for starting material (a, star label, upper panels of FIGS. 2A and 2B) and compound b (lower panels of FIGS. 2A and 2B). FIG. 2C shows the molecular weight of compound b monitored by PAGE. The M1 and M2 lanes were the same protein marker (Invitrogen; Cat. No. LC5925), except that in the M1 lane, the protein markers were illuminated with white light and in the M2 lane, the protein markers were illuminated with UV light, respectively. The 17 kDa protein maker showed red fluorescent color under the UV light. The L lane indicates a distribution of molecular weights of compound b, FIG. 2D shows a conformation plot with three fitting lines including S1 (blue line), S2 (gray line) and S3 (black line) for compound b, PEG and dendrimer, respectively.

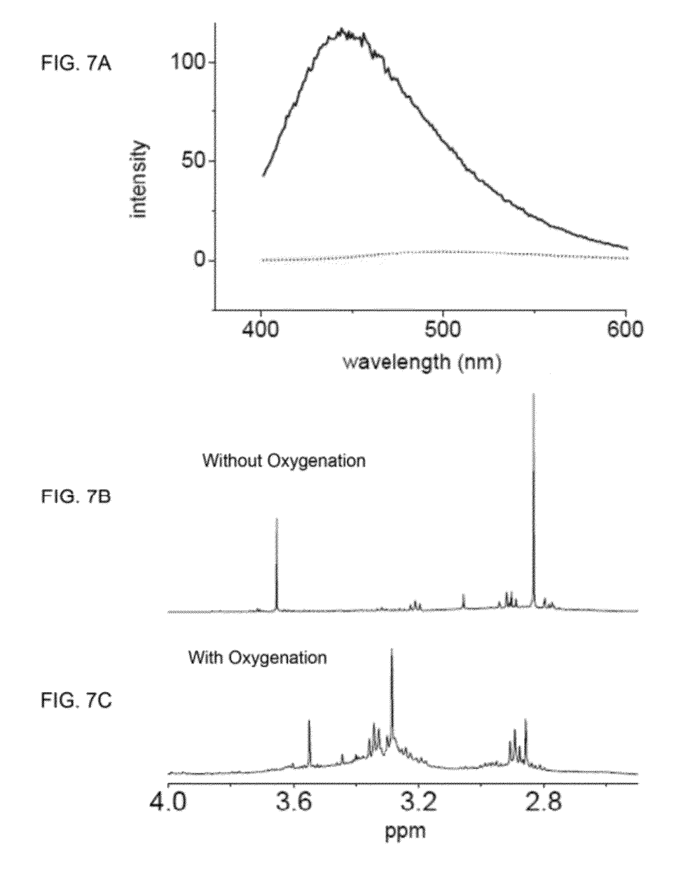

To further elucidate die mechanism of polymerization of compound b, two sets of control experiments were performed. FIG. 7A (dashed line) shows that no significant photoluminescent PEI could be observed after gassing the solution with oxygen (a well-known radical scavenger) during polymerization, indicating the absence of compound b formation. Instead, when nitrogen (an inert gas) was gassed into the solution during synthesis, an intensive photoluminescence was observed (FIG. 7A, solid line), suggesting that the photoluminescent LPEI were formed, ¹H NMR spectrum from the oxygenated solution (FIG. 7C) was compared with that from the nitrogen gassed solution (FIG. 7B). It showed several new peaks between 2.5 ppm and 4.0 ppm, and none of the peaks could be assigned to compound b. The results suggested an inhibition in the LPEI's propagation when oxygen was blown into the solution during synthesis.

FIG. 7A shows the emission spectra of LPEIs (b) in response to a typical radical scavenger (O_{2(g)}). The solid line and dashed line represents compound b formed in the presence of blowing N_{2(g)} and O_{2(g)} during synthesis, respectively, FIGS. 7B-7C are ¹H spectra (400 MHz) showing the formation of compounds b without (FIG. 7B) and with, oxygen gas (FIG. 7C) being passed into the solution during the synthesis.

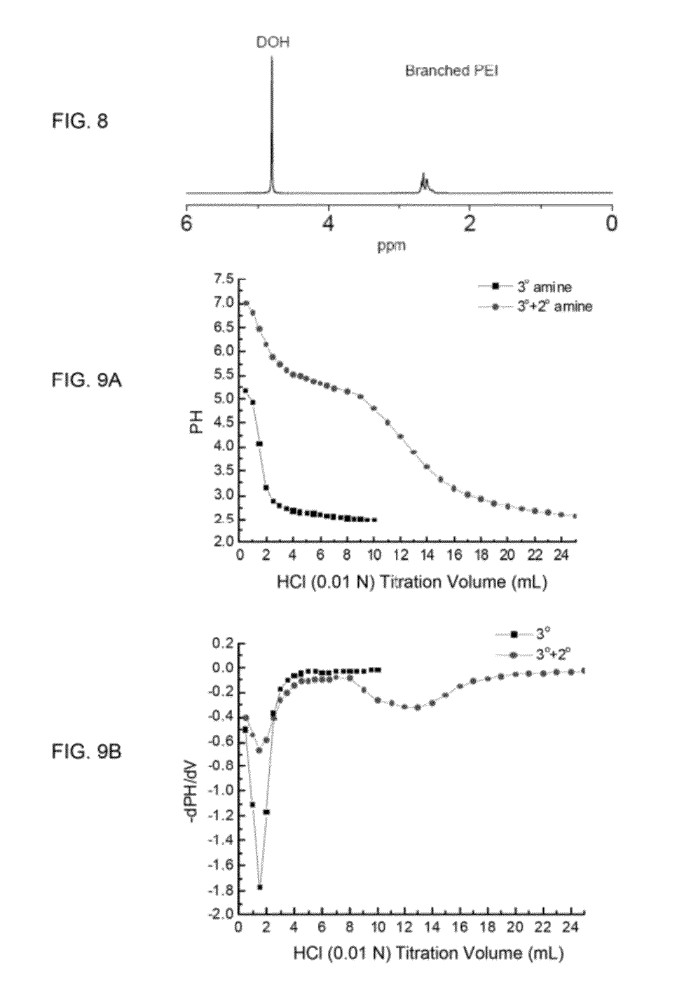

It was speculated that a plausible mechanism of polymerization was a free radical-induced self-polymerization by cyclic aliphatic amine (termed aziridine as shown in FIG. 1), which is a well-known intermediate for preparing BPFI by acid-catalyzed polymerization. A small peak at 1.56 ppm (data not shown) in the ¹H NMR spectrum was attributed to the methylene groups in aziridine. However, the amine peak of aziridine was not observed. Meanwhile, a comparison of ¹H NMR spectra between compound b (FIG. 2A) and commercial BPEI (FIG. 8) revealed a significant differences in their chemical shifts; multiple peaks appearing around 2.6˜2.7 ppm for commercial BPEI were not assigned to compound b. FIG. 8 shows ¹H spectra of commercial BPEI. To calculate the number of secondary amines (2°-NH₂) of compound b of the invention after titration (FIGS. 9A-9B), the percentage of 2°-NH₂ was ca. 90%, confirming compound b was linear-like. Moreover, while the reaction time was gradually increased, over 10 min, compound b was completely decomposed, and the emission wavelength and intensity were red shifted and decreased, respectively. Therefore, it is essential to control the reaction within 10 mm. FIG. 9A shows the titration curves of compound b, and the differential of the titration curves are shown in FIG. 9B, as measured according to a previous report (Siggia, et al. (1988) “In Quantitative Organic Analysis via Functional Groups” 4th ed.; Krieger: Malabar, Fla., pp 569-572).

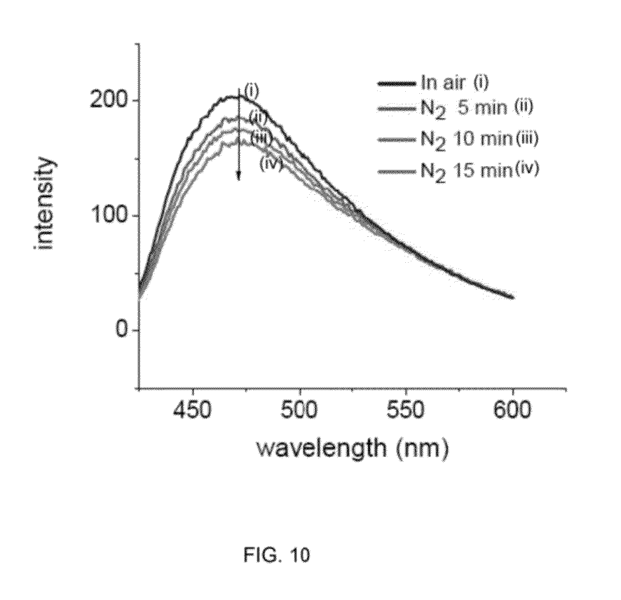

The photoluminescence intensity decreased with a decreased oxygen level in the aqueous solution (FIG. 10). FIG. 10 shows emission spectra of compound b in water, the synthesis of which being carried out in air or N_{2(g)}-bubbling of various duration. The result was consistent with a previous report, suggesting that amazing photoluminescence might be derived from the incorporation effect between oxygen in the solution and the nitrogen atoms of the polymer. Such a photoluminescent polymer has been demonstrated to act as a cellular tracker.

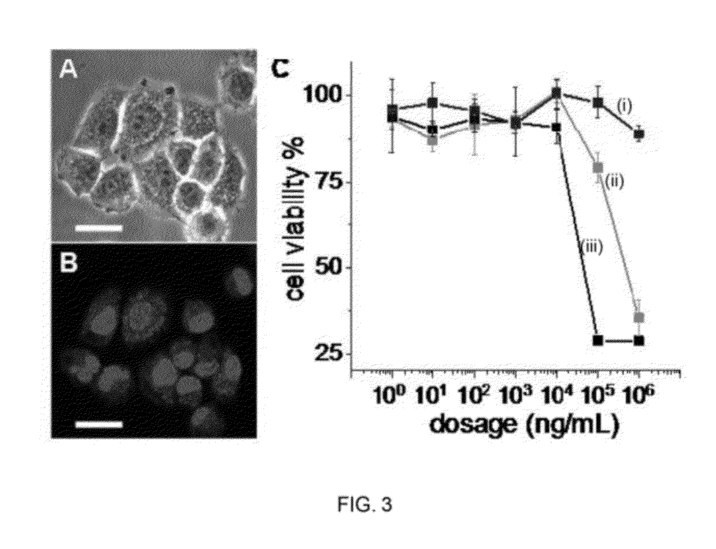

FIG. 3 shows confocal microscope images of intracellular delivery and cytotoxicity evaluation of b. H460 human lung cancer cells were treated with b for 1.5 h. FIG. 3A shows a one-color image of cells treated with compound b with the image overlapping with a phase image of the cells. FIG. 3B shows a two-color colocalization image of H460 cells treated with compound b counterstained with a specific nuclear dye (SYTO® 59). FIG. 5C show the cytotoxicity of compound b, line (i), being evaluated and compared with commercial BPEI, line (iii), and a mixture of LPEIs and BPEIs, line (ii).

The intensive blue photoluminescence in cytoplasm (FIGS. 3A and 3B) indicated that the polymers capped with positive charges were able to cross the cell membrane. In addition, the cytotoxicity of compound b and other commercial PEIs have been evaluated and compared. The results are shown in FIG. 3C. The concentration of commercial BPEIs (line iii) at 10⁵ ng/mL induced severe cell death (25% cell viability as compared with control), commercial LPEIs (line ii, 10⁵ ng/mL) also cause noticeable cell death (75% cell viability compared with the control). Commercial LPEIs at a higher concentration (10⁶ ng/mL) can also induce significant cell death (25% cell viability compared with control). On the contrary, the LPEI according to the invention showed no significant cytotoxicity at these two high concentrations (10⁵ and 10⁶ ng/mL). The decreased cytotoxicity of LPEI according to the invention might stem from the simple synthesis strategy that involved minimal hazardous chemicals such as the organic solvents.

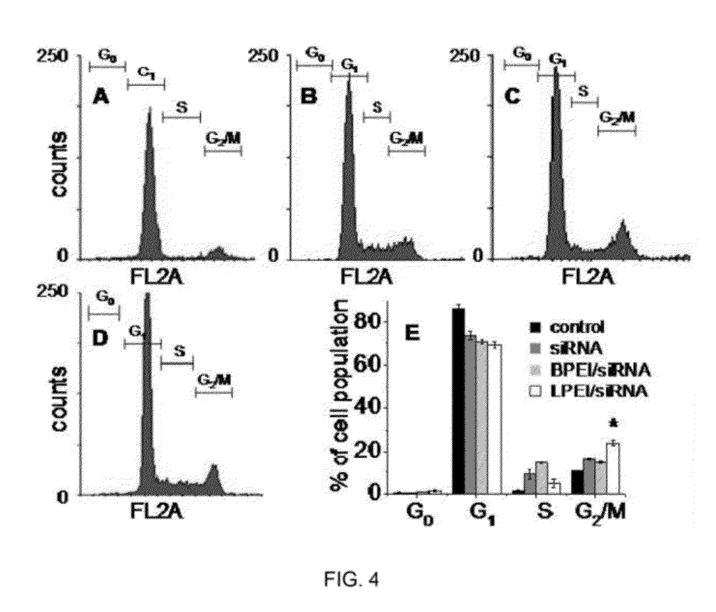

PEI has a high cationic density and can associate with nucleic acids such as siRNA to form a cationic polyplex. The efficiency of the PEI according to the invention as a delivery vehicle for siRNA was examined. Compound b (LPEI) was used to associate with a siRNA for silencing Cyclin B1, which is an indispensable protein for cell mitosis. The specific siRNA can induce cell cycle arrest and further inhibit tumor cell growth through silencing gene encoding Cyclin B1. H460 human lung cancer cells were incubated with compound b/siRNA complex ([compound b]=100 ng/mL, [siRNA]=200 nM) for 24 h (in the absence of fetal bovine serum). Flow cytometry data (FIGS. 4 A to 4C) showed that when H460 human lung cancer was treated with the compound b/siRNA complex (FIG. 4C), their counts in G2/M phase of the cell cycle were significantly enhanced as compared with the control (FIG. 4A) and siRNA alone (FIG. 4B). The population proportions of cells were analyzed and summarized in histogram profile (FIG. 4E). After treating cells with siRNA alone, the population in G2/M phase of the cell cycle was only increased to 5.52%±0.406 (compared with control). On the contrary, when cells were treated with the PEI/siRNA complex of the present invention, the population in G2/M phase of the cell cycle was significantly increased to 13.70%±0.896 (p<0.05, n=3) as compared with the control. These results indicated that the cell mitosis was efficiently arrested in G2/M phase of cell cycles by the treatment of compound b/siRNA complex. Meanwhile, cells were also incubated with commercial BPEI/siRNA complex (FIGS. 4D-4E); no discernible difference of population in G2/M phase was observed between the treatment of siRNA alone (5.52%±0.406 compared with control) and commercial BPEI/siRNA (4.00%±0.377 compared with the control). The results indicated, that LPEI of the invention was an excellent vehicle for siRNA delivery. Taking the safety into consideration, it is a reasonable expectation that the dosage of compound b in an animal model can be increased when a higher amount of siRNA is need for better efficacy.

FIG. 4 shows the results of cell cycle arrest analysis. H460 lung cancer cells were treated with compound b/siRNA complex for 24 h, and the cell cycle distribution was examined by flow cytometry. (A) Control; (B) siRNA alone; (C) compound b/siRNA; (D) commercial BPEI/siRNA; and (E) the histogram profile of cell cycle arresting analysis. FL2A: fluorescence pulse area. *p<0.05.

In summary, the invention relates to a one-pot and catalyst/organic solvent free reaction under synchrotron X-ray irradiation to synthesize photoluminescent LPEI from ethylenediamine. The LPEI could penetrate the cell membrane, enter the cytoplasm, and had much less cytotoxic effect when compared with BPEI and commercial LPEI. The LPEI can be used for efficient si RNA delivery. This synthetic strategy may accelerate the biological applications of LPEI in the future.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

What is claimed is:

1. A method of synthesizing polyethyleneimine with a substantially linear backbone comprising:
exposing ethylenediamine dissolved in a solution to electromagnetic radiation for a sufficient time to polymerize the ethylenediamine and thereby resulting in formation of polyethylenimine with a substantially linear backbone in the solution.

2. The method of claim 1, wherein the ethylenediamine is irradiated at a temperature between 4° C. and 60° C.

3. The method of claim 1, wherein the ethylenediamine is irradiated for no more than 10 minutes.

4. The method of claim 1, wherein the polyethylenimine with a substantially linear backbone is formed in the absence of a reagent chosen from organic solvents, acids, and poly(alkyl ethylene).

5. The method of claim 1, wherein the polyethylenimine with a substantially linear backbone has a distribution of molecular weights ranging from 1 kDa to 200 kDa.

6. The method of claim 1, wherein the electromagnetic radiation is selected from X-rays, microwaves, and gamma-rays.

7. The method of claim 1, wherein the electromagnetic radiation comprises X-rays.

8. The method of claim 1, wherein the radiation has energy of 4 to 3,000 KeV and a radiation dose of from 2×10³ to 10 Gy/s.

9. The method of claim 1, wherein the polyethylenimine with a substantially linear backbone is formed with stirring.

10. The method of claim 1, wherein the polyethylenimine with a substantially linear backbone is formed without stirring.

11. The method of claim 1, further comprising removing ethylenediamine dissolved in the solution after formation of the polyethyleneimine with a substantially linear backbone.

12. A composition comprising:
polyethylenimine with a substantially linear backbone in an effective amount, the polyethylenimine being synthesized from exposing, ethylenediamine to electromagnetic radiation for a sufficient time according to the method of claim 11, wherein the polyethylenimine comprises a backbone conformation that is substantially linear and has a distribution of molecular weights (MW) ranging from 1 kDa to 200 kDa; and wherein the polyethyleneimine has no cytotoxicity at a concentration of 12 μg/ml.

13. The composition of claim 12, comprising polyethyleneimine having a concentration ranging from 100 ng/ml to 10⁶ ng/ml, and wherein the polyethyleneimine has no significant cytotoxicity in the concentration range indicated.

14. The composition of claim 12, comprising polyethyleneimine having a concentration ranging from 100 ng/ml to 10⁵ ng/ml; and wherein the polyethyleneimine has no significant cytotoxicity in the concentration range, indicated.

15. The composition of claim 12, further comprising a nucleic acid.

16. The composition of claim 15, wherein the nucleic acid is a small interfering RNA (siRNA).

17. A method of delivering a nucleic acid into a cell in vivo comprising exposing the cell in vivo to a composition according to claim 15.

18. A method of synthesizing a homopolymer with a substantially linear backbone, comprising:
exposing a monomer dissolved in a solution to electromagnetic radiation for a sufficient time to polymerize the monomer and thereby resulting in formation of a homopolymer with a substantially linear backbone in the solution.

19. The method of claim 18, wherein the electromagnetic radiation has energy of 4 KeV to 3,000 KeV and a radiation dose of 2×10³ Gy/s to 10⁷ Gy/s.

20. The method of claim 18, wherein the monomer and homopolymer are without double and triple bonds; and Wherein the method is without the step of precipitating the homopolymer from a polymer radical; and the radiation is unfiltered through a means for screening or is unfiltered through a radiation mask.

21. The composition of claim 12, wherein the polyethylenimine with a substantially linear backbone has a distribution of molecular weights ranging from 1 kDa to 30 kDa.
